(19) United States
(12) Patent Application Publication
(10) Pub. No.: US 2024/0306903 A1
(43) Pub. Date: Sep. 19, 2024

(54) ENDOSCOPE

(71) Applicant: FUJIFILM Corporation, Tokyo (JP)

(72) Inventors: Ukyo YAGYU, Ashigarakami-gun (JP); Hidefumi AKAHANE, Ashigarakami-gun (JP)

(73) Assignee: FUJIFILM Corporation, Tokyo (JP)

(21) Appl. No.: 18/599,142

(22) Filed: Mar. 7, 2024

(30) Foreign Application Priority Data
Mar. 17, 2023 (JP) 2023-043369

(51) Int. Cl. A61B 1/018; A61B 1/00; A61B 1/005; A61B 1/12

(52) U.S. Cl. CPC A61B 1/018; A61B 1/00091; A61B 1/00165; A61B 1/005; A61B 1/126

(57) ABSTRACT

An endoscope includes an insertion part incorporating a treatment tool pipe line, an imager, and a part of a cable connected to the imager, a distal end portion of the insertion part has a tubular first member as defined herein, a first housing and a second housing are provided adjacent to each other at a proximal end portion of the first member as defined herein, an opening communicating with the second housing is formed at an outer peripheral surface of the proximal end portion, in a view in an axial direction of the insertion part, the outer peripheral surface of the proximal end portion includes a first region and a second region as defined herein, in the view, the second region and the opening are disposed on opposite sides with the center of the insertion part interposed therebetween, and the first housing is disposed adjacent to the second region.

### CROSS-REFERENCE TO RELATED APPLICATIONS

The present application claims priority under 35 U.S.C. § 119 to Japanese Patent Application No. 2023-043369, filed on Mar. 17, 2023. The above application is hereby expressly incorporated by reference, in its entirety, into the present application.

### BACKGROUND OF THE INVENTION

### 1. Field of the Invention

The present invention relates to an endoscope.

### 2. Description of the Related Art

JP2022-55802A discloses an endoscope comprising an insertion part having a round pipe shape to be inserted into a body cavity, a treatment tool path that has one end open to a distal end surface of the insertion part, penetrates the insertion part in a direction of an axial length, and through which a treatment tool passes, and a fluid path that has one end open to the distal end surface of the insertion part, penetrates the insertion part in the direction of the axial length, and through which a fluid that has flowed from the one end flows, in which the treatment tool path and the fluid path are partially continuous in a cross-sectional view.

JP2001-78953A discloses an endoscope having a substantially cylindrical shape and including an observation lens and a cleaning nozzle that cleans a distal end surface of the observation lens at a distal end portion thereof, in which an irregular portion that is located at an outer peripheral portion of the distal end portion of the endoscope and in the vicinity of a line connecting the observation lens and the cleaning nozzle and that protrudes to the outside with respect to a diameter of a circle at the other outer peripheral portion is provided.

JP2012-110526A discloses a distal end portion structure of an endoscope insertion part comprising an imaging unit that is incorporated in a distal end portion of an endoscope, and a light guide bundle that is branched into a plurality of parts around the imaging unit and that transmits illumination light, in which a treatment tool channel, an air/water supply channel, and a forward water supply channel are disposed in the distal end portion.

### SUMMARY OF THE INVENTION

An object of the technology of the present disclosure is to provide an endoscope capable of reducing a burden on both a subject and a user.

An endoscope according to an aspect of the present disclosure comprises an insertion part, in which the insertion part incorporates a treatment tool pipe line into which a treatment tool is inserted, an imaging unit, and a part of a cable connected to the imaging unit, a distal end portion of the insertion part has a tubular first member that houses an incorporated object of the insertion part, a first housing part that houses the treatment tool pipe line and a second housing part that houses the imaging unit are provided adjacent to each other at a proximal end portion of the first member, an opening communicating with the second housing part is formed on an outer peripheral surface of the proximal end portion, in a state of being viewed in an axial direction of the insertion part, the outer peripheral surface of the proximal end portion includes a first region where a distance from a center of the insertion part is a first distance, and a second region where the distance is a second distance that is larger than the first distance, in a state of being viewed in the axial direction, the second region and the opening are disposed on opposite sides with the center of the insertion part interposed therebetween, and the first housing part is disposed adjacent to the second region.

An endoscope according to an aspect of the present disclosure comprises an insertion part, in which the insertion part incorporates a treatment tool pipe line into which a treatment tool is inserted, and a ratio of an inner diameter of the treatment tool pipe line to an outer diameter of a distal end portion of the insertion part is 35% or more, and the inner diameter is 3 mm or more.

According to the present disclosure, it is possible to provide an endoscope capable of reducing a burden on both a subject and a user.

### BRIEF DESCRIPTION OF THE DRAWINGS

FIG. 1 is a schematic view showing an endoscope 1 according to an embodiment of the technology of the present disclosure.

FIG. 2 is a view of an insertion part 2 as viewed from a distal end side in an axial direction.

FIG. 3 is an enlarged perspective view of a distal end portion 7 and a bendable portion 6 of the endoscope 1.

FIG. 4 is a view showing an internal structure of the bendable portion 6 shown in FIG. 3.

FIG. 5 is a partially enlarged view omitting the bendable portion 6 shown in FIG. 3.

FIG. 6 is a partially enlarged view omitting the bendable portion 6 shown in FIG. 3 and as viewed from a direction different from that of FIG. 5.

FIG. 7 is a partially enlarged exploded perspective view omitting the bendable portion 6 shown in FIG. 3 and as viewed from the distal end side.

FIG. 8 is a schematic view partially showing a cross section taken along line A-A shown in FIG. 2.

FIG. 9 is a perspective view of a first member 71 constituting the distal end portion 7 as viewed from a proximal end side.

FIG. 10 is a perspective view of the first member 71 constituting the distal end portion 7 as viewed from the distal end side.

FIG. 11 is a view of the first member 71 constituting the distal end portion 7 as viewed from the proximal end side in the axial direction.

FIG. 12 is a view of the first member 71 constituting the distal end portion 7 as viewed from the distal end side in the axial direction.

FIG. 13 is a side view of a bending piece portion 60 as viewed in a right direction.

FIG. 14 is an exploded perspective view of two adjacent annular members 64 in the bending piece portion 60.

FIG. 15 is a view of a connection state of the two annular members 64 shown in FIG. 14 as viewed from the proximal end side in the axial direction.

FIG. 16 is a schematic view showing a disposition of incorporated objects that are incorporated in the bending piece portion 60.

### DESCRIPTION OF THE PREFERRED EMBODIMENTS

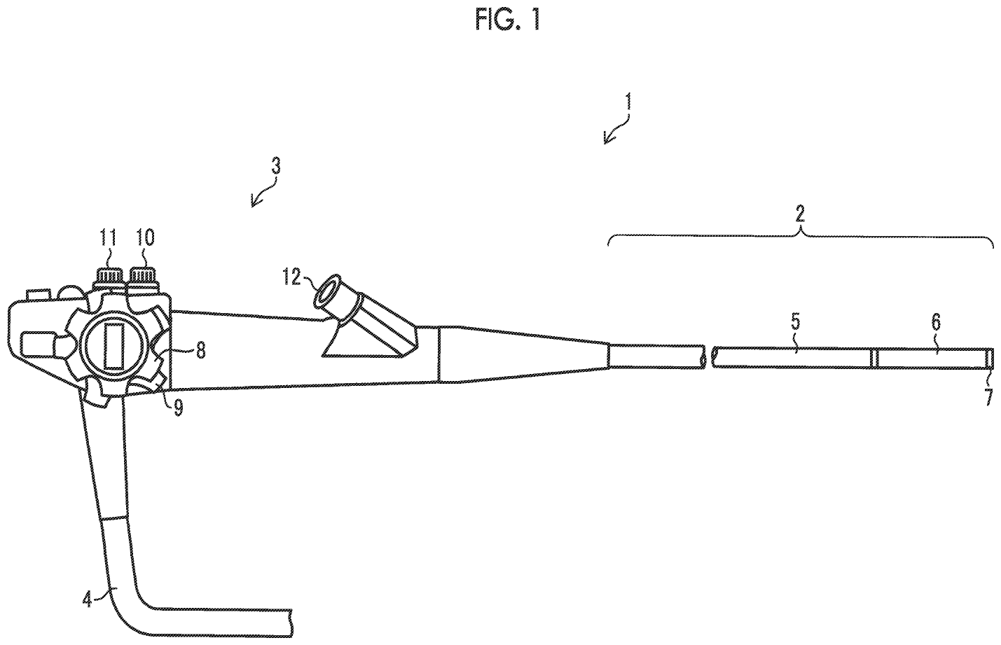

FIG. 1 is a schematic view showing an endoscope 1 according to an embodiment of the technology of the present disclosure. The endoscope 1 in FIG. 1 comprises an elongated insertion part 2 that is inserted into a subject, an operating part 3 that is connected to a proximal end of the insertion part 2 and that is used for gripping and operating the endoscope 1, and a universal cord 4 that connects the endoscope 1 to a system constituent device such as a light source device and a processor device (not shown). The endoscope 1 according to the present embodiment is an upper endoscope for observing a stomach, a duodenum, or the like, a lower endoscope (colonoscope) for observing a large intestine or the like, or the like.

The insertion part 2 is composed of a soft portion 5, a bendable portion 6, and a distal end portion 7 that are consecutively provided in order from the proximal end toward a distal end. The soft portion 5 is flexible and is bendable in any direction along an insertion path of the insertion part 2. The operating part 3 is provided with angle knobs 8 and 9, a treatment tool inlet port 12, an air/water supply button 10, a suction button 11, and the like.

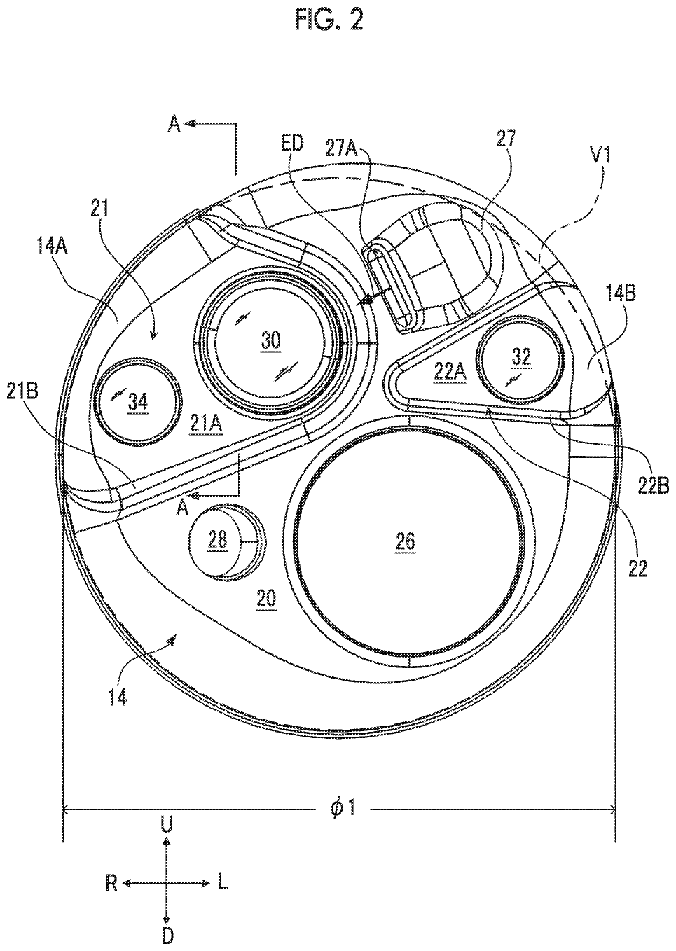

The bendable portion 6 is bent in each of up-down and left-right directions by an operation of each of the angle knobs 8 and 9. A treatment tool, such as a forceps, is inserted from the treatment tool inlet port 12 and is led out from a forceps port 26 (see FIG. 2) provided in the distal end portion 7. In addition, the distal end portion 7 is provided with an observation window 30 (see FIG. 2) for imaging a site to be observed in a body, and a first illumination window 32 and a second illumination window 34 (see FIG. 2) for irradiating the site to be observed with illumination light.

The insertion part 2 is inserted into a subject by moving along an axial direction thereof, and the angle knobs 8 and 9 of the operating part 3 are rotationally operated to bend the bendable portion 6 of the insertion part 2 in the up-down and left-right directions. Accordingly, the distal end portion 7 of the insertion part 2 can be directed to a desired direction in the body, and an observation image can be acquired by the observation window 30 provided in the distal end portion 7.

FIG. 2 is a view of the insertion part 2 as viewed from a distal end side in the axial direction. In FIG. 2, as directions in which the bendable portion 6 can be bent, an upper direction U, a lower direction D opposite to the upper direction U, a right direction R, which is one direction of directions orthogonal to the upper direction U and the lower direction D, and a left direction L opposite to the right direction R are shown.

As shown in FIG. 2, the distal end portion 7 has a distal end surface 14 disposed on the most distal end side. The distal end surface 14 is formed in a circular shape as viewed in the axial direction. In FIG. 2, an imaginary circle V1 is shown. The imaginary circle V1 is a perfect circle centered at a center of the insertion part 2 in a case of being viewed in the axial direction. An outer peripheral edge of the distal end surface 14 has, as a whole, a shape along an outer periphery of the imaginary circle V1. A portion that occupies more than half of the outer peripheral edge of the distal end surface 14 (a range from about 3 o'clock to about 11 o'clock in an example of the figure clockwise in a case where a position on a circumference of the imaginary circle V1 is represented by a time of a clock) has a shape overlapping with an outer peripheral edge of the imaginary circle V1, and a portion excluding the portion (a range from about 11 o'clock to about 3 o'clock clockwise) has a shape that bulges to the outside of the imaginary circle V1. In this way, in the endoscope 1, the portion that occupies more than half of the outer peripheral edge of the distal end surface 14 is an arc of the perfect circle (imaginary circle V1). A diameter of the imaginary circle V1 is defined as an outer diameter φ1 of the distal end portion 7.

The distal end surface 14 is located on a distal end side with respect to the outer peripheral edge thereof, and comprises a first surface 20 that is formed inside the outer peripheral edge of the distal end surface 14 and that is perpendicular to the axial direction, a second surface 21 that protrudes from the first surface 20 toward the distal end side, and a third surface 22 that protrudes from the first surface 20 toward the distal end side. An outer peripheral edge of the first surface 20 and the outer peripheral edge of the distal end surface 14 are connected to each other by an inclined surface.

The second surface 21 includes a flat surface 21A perpendicular to the axial direction, and a stepped portion 21B which is a portion connecting the flat surface 21A and the first surface 20. The stepped portion 21B is composed of an inclined surface that rises from the first surface 20 toward the flat surface 21A. The flat surface 21A and the outer peripheral edge of the distal end surface 14 are connected to each other by an inclined surface 14A.

The third surface 22 includes a flat surface 22A perpendicular to the axial direction, and a stepped portion 22B which is a portion connecting the flat surface 22A and the first surface 20. The stepped portion 22B is composed of an inclined surface that rises from the first surface 20 toward the flat surface 22A. The flat surface 22A and the outer peripheral edge of the distal end surface 14 are connected to each other by an inclined surface 14B.

The first surface 20 is provided with a forceps port 26, a nozzle 27 for fluid jetting, and a water jetting (WJ: front water jetting) nozzle 28.

As viewed from the axial direction, the second surface 21 extends from the outer peripheral edge of the distal end surface 14 toward the nozzle 27 and has a substantially triangular shape. The second surface 21 is separated from the forceps port 26 and the nozzle 27. On the flat surface 21A of the second surface 21, the observation window 30 and the second illumination window 34 are disposed. In the flat surface 21A, the observation window 30 is disposed on a side of the nozzle 27, and the second illumination window 34 is disposed on a side opposite to the nozzle 27 with the observation window 30 interposed therebetween.

As viewed from the axial direction, the third surface 22 extends from the outer peripheral edge of the distal end surface 14 toward the second surface 21 and is located between the forceps port 26 and the nozzle 27. On the flat surface 22A of the third surface 22, the first illumination window 32 is disposed.

The forceps port 26 communicates with the treatment tool inlet port 12 via a treatment tool pipe line (a treatment tool pipe line 260 which will be described later), and the treatment tool inserted from the treatment tool inlet port 12 is led out from the forceps port 26. In addition, the forceps port 26 is connected to a suction pump (negative pressure source) via the treatment tool pipe line. By operating the suction button 11, cleaning water and adherent substances (blood or the like in the subject) are sucked from the forceps port 26.

The nozzle 27 comprises a jetting port 27A for jetting a fluid (a liquid or a gas), and the jetting port 27A is directed toward the observation window 30. The jetting port 27A of the nozzle 27 jets the fluid such as a liquid or a gas to a surface of the observation window 30 and to a peripheral portion thereof in a jetting direction ED indicated by an arrow and dispels and removes the adherent substances, the cleaning water, or the like remaining on the observation window 30 located on a side in the jetting direction ED. The nozzle 27 communicates with a fluid pipe line (a fluid pipe line 270 which will be described later) disposed in the insertion part 2, the operating part 3, and the universal cord 4, and jets a fluid sent from an air/water supply device (not shown), to the observation window 30.

The WJ nozzle 28 jets a liquid such as cleaning water or a chemical liquid toward the site to be observed. The WJ nozzle 28 communicates with a fluid pipe line (a liquid pipe line 280 which will be described later) disposed in the insertion part 2, the operating part 3, and the universal cord 4, and directly sprays the liquid sent from a liquid supply device (not shown), to the site to be observed. The WJ nozzle 28 is disposed at a position adjacent to the forceps port 26 on the first surface 20.

The observation window 30 is a constituent element of an observation unit that acquires an image of the site to be observed in order to observe the inside of the subject and introduces subject light from the site to be observed to an optical system (a lens group or the like) and to an imaging element which are other constituent elements of the observation unit. The image captured by the observation unit is sent as an observation image to a processor device connected by the universal cord 4.

The first illumination window 32 and the second illumination window 34 are constituent elements of an illumination unit mounted on the distal end portion 7, and irradiate the site to be observed with illumination light emitted from a light emitting unit which is another constituent element of the illumination unit. The illumination light emitted from the light emitting unit propagates through a light guide that is inserted through the inside of the endoscope 1 from a light source device connected by the universal cord 4.

Hereinafter, an example of an internal structure of the insertion part 2 will be described in detail.

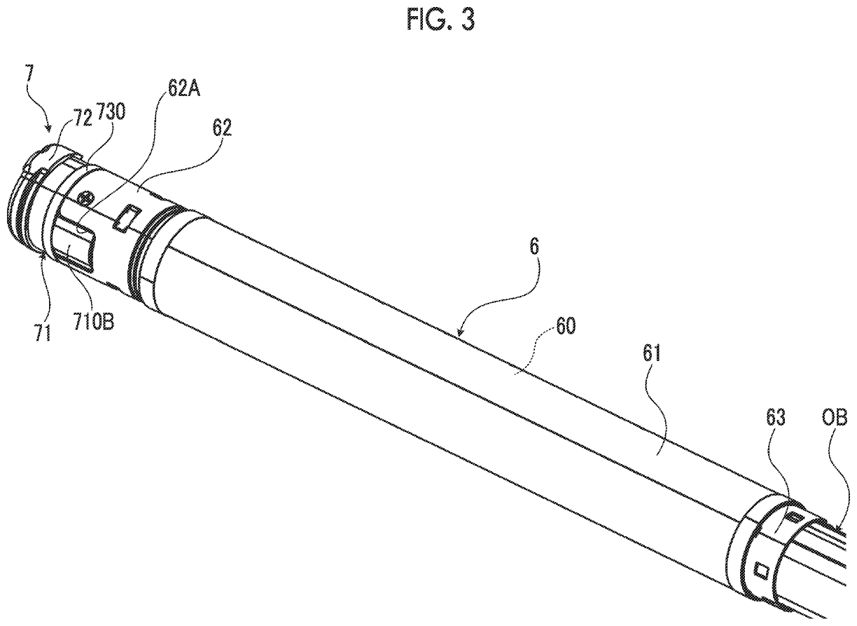

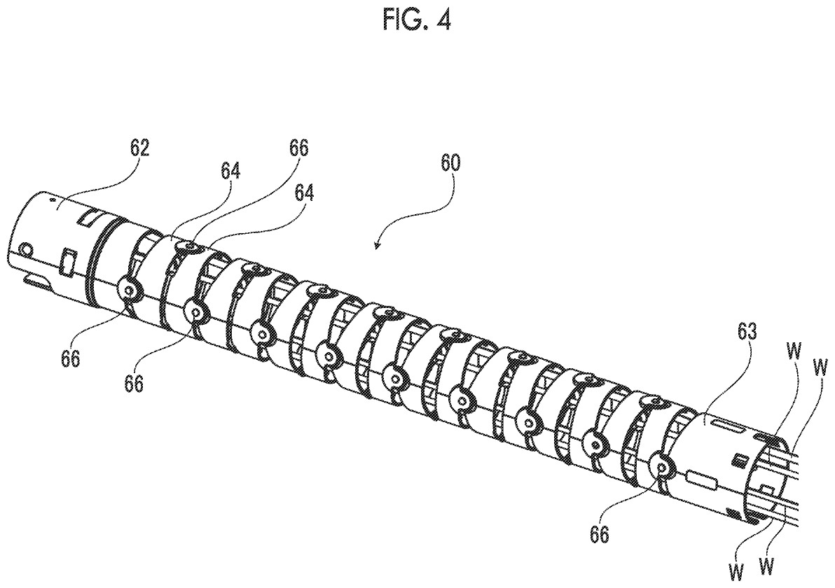

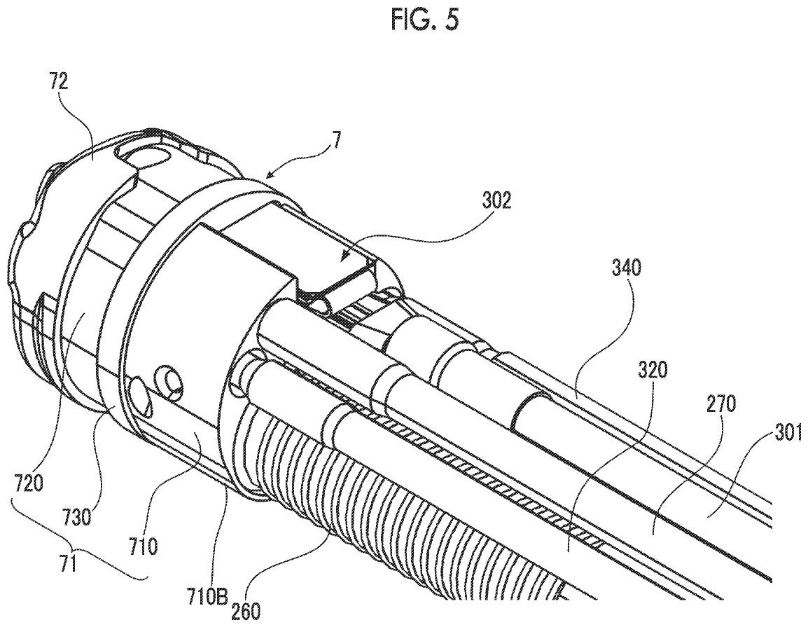

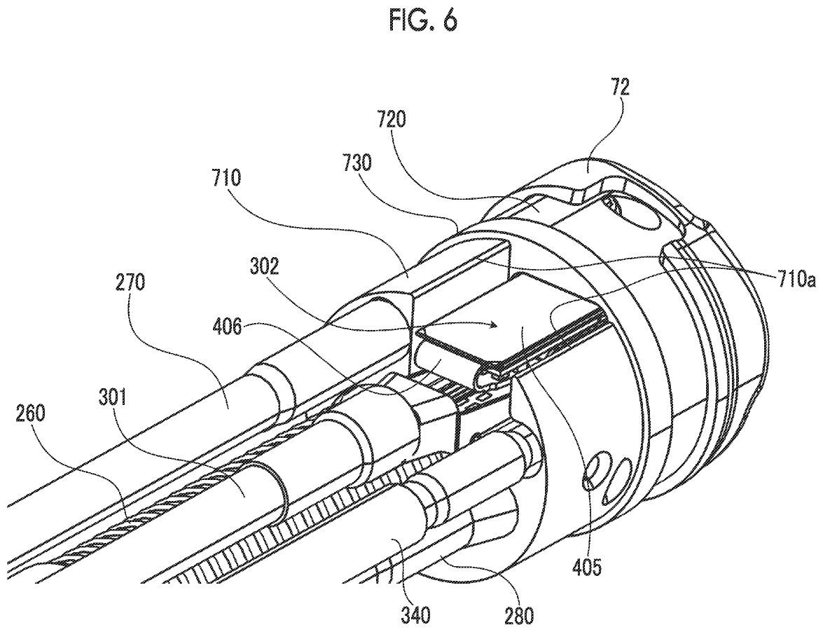

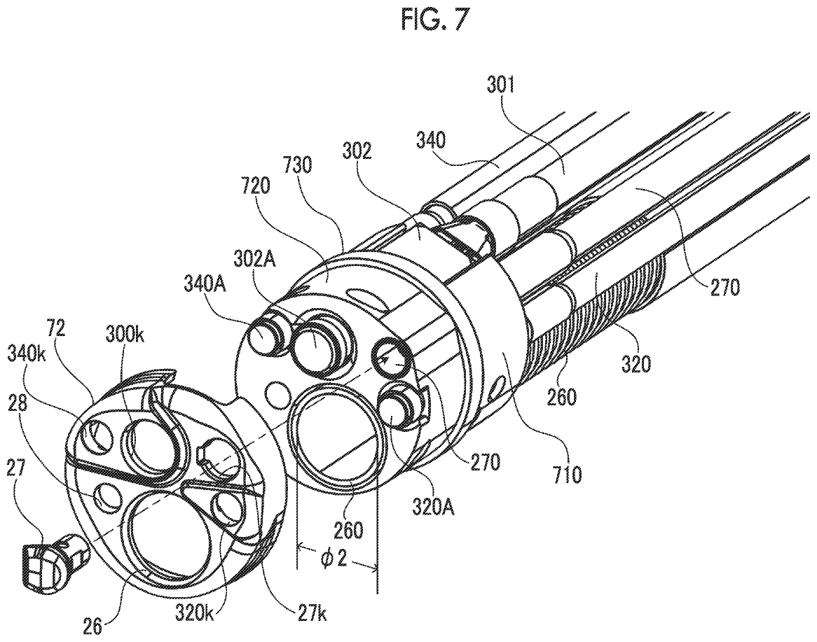

FIG. 3 is an enlarged perspective view of the distal end portion 7 and the bendable portion 6 of the endoscope 1. FIG. 4 is a view showing an internal structure of the bendable portion 6 shown in FIG. 3. FIG. 5 is a partially enlarged view omitting the bendable portion 6 shown in FIG. 3. FIG. 6 is a partially enlarged view omitting the bendable portion 6 shown in FIG. 3 and as viewed from a direction different from that of FIG. 5. FIG. 7 is a partially enlarged exploded perspective view omitting the bendable portion 6 shown in FIG. 3 and as viewed from the distal end side.

As shown in FIGS. 5 and 6, the distal end portion 7 comprises a first member 71, which is a distal end portion body that has a tubular shape with an opening 710a formed on a proximal end side of an outer peripheral surface, and a second member 72, which is a distal end cap that has a tubular shape and that is fixed to the outer peripheral surface of the first member 71 on a distal end side. An axial direction of the first member 71 and the second member 72 coincides with the axial direction of the insertion part 2. A distal end of the second member 72 constitutes the distal end surface 14 described above. The first member 71 is made of, for example, metal. The second member 72 is made of, for example, a resin. In the present specification, a tubular member refers to a member in which one or more spaces are included therein, the spaces being connected from one end surface to the other end surface of the member in an axial direction.

As shown in FIG. 5, the first member 71 comprises an annular large-diameter portion 730 that is provided substantially at a central portion between a proximal end and a distal end and that has the largest outer diameter among the first members 71, a proximal end portion 710 that extends from a proximal end-side end surface of the large-diameter portion 730 to the proximal end side, and a member distal end portion 720 that extends from a distal end-side end surface of the large-diameter portion 730 to the distal end side. The second member 72 is fixed to a distal end side of an outer peripheral surface of the member distal end portion 720. In a state of being viewed in the axial direction, the large-diameter portion 730 constitutes a convex portion that protrudes from outer peripheral surfaces of the proximal end portion 710 and the member distal end portion 720, and is provided along a circumferential direction of the first member 71.

As shown in FIG. 3, the bendable portion 6 comprises a bending piece portion 60 provided inside a coating member (net) 61 having flexibility. As shown in FIG. 4, the bending piece portion 60 is configured by connecting a plurality of annular members 64 to be rotatable to each other. The adjacent annular members 64 are rotatably connected to each other by a caulking pin 66. On the most distal end side of the bending piece portion 60, a tubular fixing member 62 for connecting the first member 71 and the bendable portion 6 to each other is provided. The distal end portion 7 and the bendable portion 6 are connected to each other by fixing the fixing member 62 to the first member 71 by screwing or the like. The fixing member 62 and the annular member 64 located on the most distal end side among the plurality of annular members 64 are connected to be rotatable to each other by the caulking pin 66. On the most proximal end side of the bending piece portion 60, a tubular connecting member 63 that connects the soft portion 5 and the bendable portion 6 to each other is provided. The connecting member 63 and the annular member 64 located on the most proximal end side among the plurality of annular members 64 are connected to be rotatable to each other by the caulking pin 66.

Four wires W are inserted into the fixing member 62, the plurality of annular members 64, and the connecting member 63. One end side of each wire W is fixed to the fixing member 62, and the other end side of each wire W is fixed to an end portion of a chain wound around a sprocket (not shown) that is rotationally operated by the angle knobs 8 and 9. Thus, the bendable portion 6 is remotely operated to be bent (angle operation) by rotating the angle knobs 8 and 9 provided in the operating part 3.

As shown in FIG. 3, an incorporated object group OB including a plurality of elongated incorporated objects is incorporated in the insertion part 2 from the proximal end to the distal end. The incorporated object group OB includes the four wires W (see FIG. 4), an imaging unit 302 and a part of a cable 301 (see FIG. 5) connected to the imaging unit 302, a left light guide 320 and a right light guide 340 (see FIG. 5) each configured by bundling a plurality of optical fibers, the treatment tool pipe line 260 (see FIG. 5) into which the treatment tool such as a forceps or a puncture needle is inserted, the fluid pipe line 270 (see FIG. 5) through which a liquid or a gas flows, and the liquid pipe line 280 (see FIG. 6) through which a liquid flows.

The cable 301, the left light guide 320, and the right light guide 340 are provided to extend from the distal end portion 7 to a proximal end of the universal cord 4. The treatment tool pipe line 260, the fluid pipe line 270, and the liquid pipe line 280 are provided to extend from the distal end portion 7 to the operating part 3.

As shown in FIG. 7, a distal end surface 302A of the imaging unit 302 is inserted into a through-hole 300k provided in the second member 72 to penetrate therethrough in the axial direction, thereby forming the observation window 30. A distal end surface 320A of the left light guide 320 is inserted into a through-hole 320k provided in the second member 72 to penetrate therethrough in the axial direction, thereby forming the first illumination window 32. A distal end surface 340A of the right light guide 340 is inserted into a through-hole 340k provided in the second member 72 to penetrate therethrough in the axial direction, thereby forming the second illumination window 34. The treatment tool pipe line 260 communicates with the forceps port 26 provided in the second member 72 to penetrate therethrough in the axial direction. The fluid pipe line 270 communicates with a through-hole 27k provided in the second member 72 to penetrate therethrough in the axial direction. The nozzle 27 is inserted into and fixed to the through-hole 27k. The liquid pipe line 280 communicates with the WJ nozzle 28 provided in the second member 72 to penetrate therethrough in the axial direction.

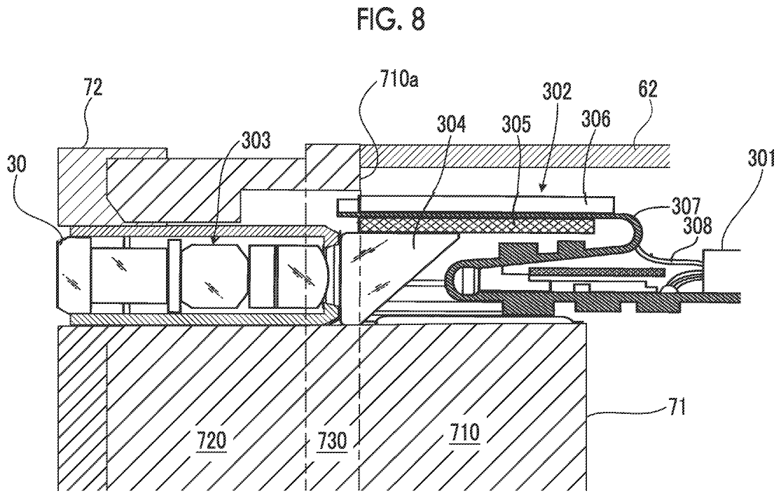

FIG. 8 is a schematic view partially showing a cross section taken along line A-A shown in FIG. 2. As shown in FIG. 8, the imaging unit 302 includes a lens barrel that houses a lens group 303 connected to the observation window 30 as an objective lens, a prism 304 that is disposed on a proximal end side of the lens barrel, an imaging element 305 that is fixed to a light emission surface of the prism 304, a main board 306 to which the imaging element 305 is attached, and a sub-board 307 to which a component or the like that cannot be attached to the main board 306 is attached. A multi-core cable is used as the cable 301. The cable 301 includes a plurality of strands 308, and the plurality of strands 308 are electrically connected to the imaging unit 302. The imaging element 305 is disposed such that a rear surface (a surface opposite to a surface fixed to the prism 304) thereof faces the opening 710a of the first member 71. In other words, the imaging element 305 is disposed in a state where the rear surface thereof faces a radial outer side of the distal end portion 7.

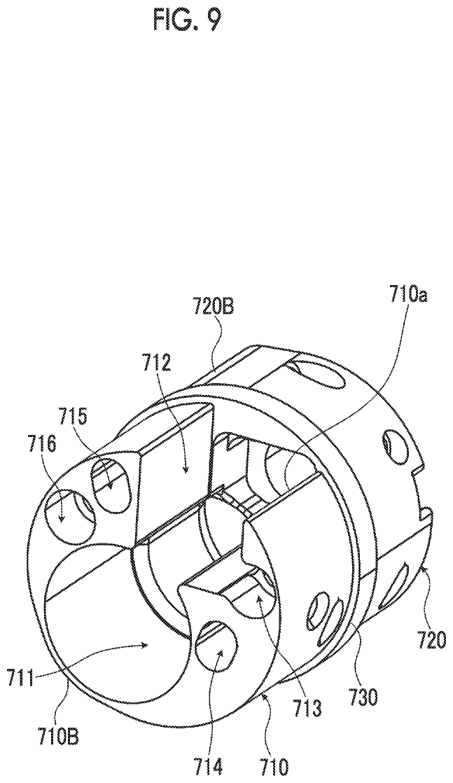

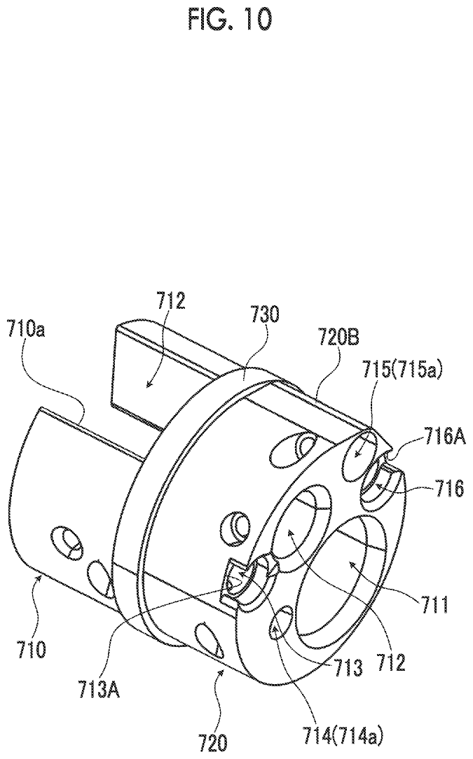

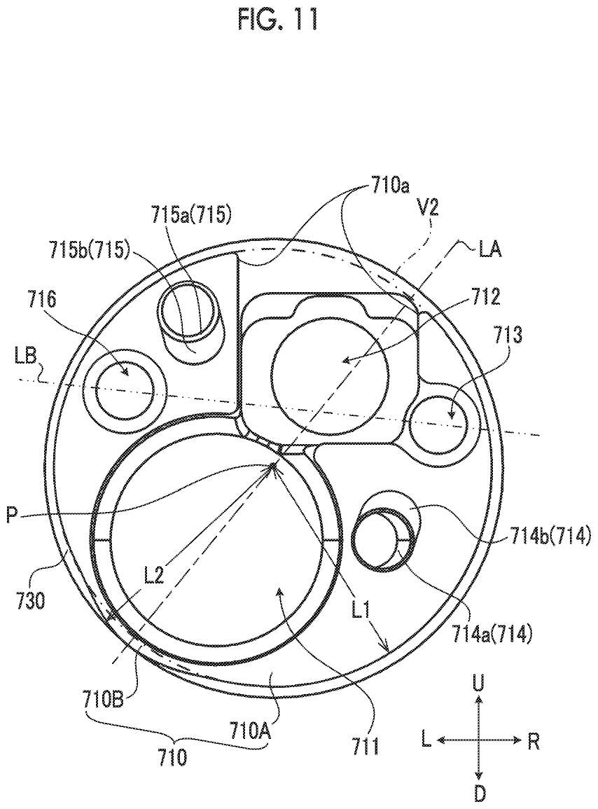

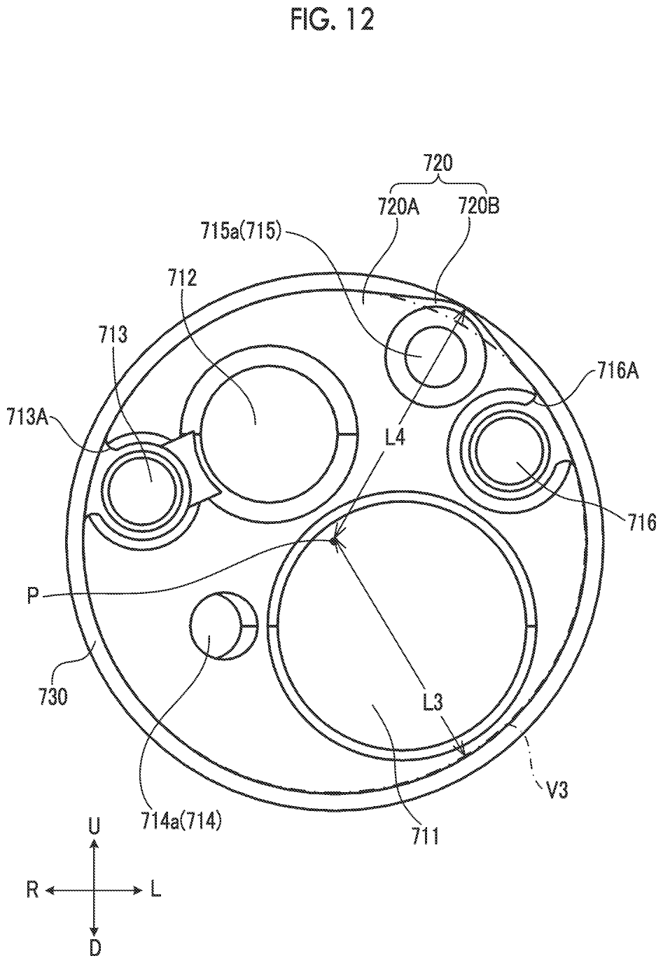

FIG. 9 is a perspective view of the first member 71 constituting the distal end portion 7 as viewed from the proximal end side. FIG. 10 is a perspective view of the first member 71 constituting the distal end portion 7 as viewed from the distal end side. FIG. 11 is a view of the first member 71 constituting the distal end portion 7 as viewed from the proximal end side in the axial direction. FIG. 12 is a view of the first member 71 constituting the distal end portion 7 as viewed from the distal end side in the axial direction.

As shown in FIGS. 9 and 11, the proximal end portion 710 has the opening 710a formed on the outer peripheral surface thereof, and has a substantially C-shape in a state of being viewed in the axial direction. In FIG. 11, an imaginary circle V2 that is concentric with a circle formed by an outer peripheral edge of the large-diameter portion 730 and that has a smaller diameter than that of the circle is shown.

The imaginary circle V2 is a perfect circle centered at a center P of the insertion part 2 in a case of being viewed in the axial direction. The proximal end portion 710 comprises a body part 710A located inside the imaginary circle V2, and a bulging portion 710B that bulges from the body part 710A to the outside of the imaginary circle V2. An outer peripheral edge of the body part 710A has a shape overlapping with an outer peripheral edge of the imaginary circle V2, and an outer peripheral edge of the bulging portion 710B has a shape that bulges to the outside of the imaginary circle V2. As shown in FIG. 5, the bulging portion 710B is provided to extend from the large-diameter portion 730 to a proximal end surface of the proximal end portion 710. As shown in FIG. 11, on an outer peripheral surface of the body part 710A, a distance from the center P is a distance L1 that is the same as a radius of the imaginary circle V2, and on an outer peripheral surface of the bulging portion 710B, the distance from the center P is a distance L2 that is larger than the distance L1.

The first member 71 is provided with a housing part 711 that houses the treatment tool pipe line 260, a housing part 712 that houses the imaging unit 302, a housing part 713 that houses the right light guide 340, a housing part 714 that houses the liquid pipe line 280, a housing part 715 that houses the fluid pipe line 270, and a housing part 716 that houses the left light guide 320. The housing part 712 may include a connection portion between the imaging unit 302 and the cable 301. All of these housing parts are each composed of a hole portion that communicates from a proximal end-side end surface to a distal end-side end surface of the first member 71. As shown in FIG. 7, the treatment tool pipe line 260, the fluid pipe line 270, the imaging unit 302, the left light guide 320, and the right light guide 340 are, respectively, in a state where distal ends thereof are at the same position as a distal end surface of the member distal end portion 720 or on the distal end side of the distal end surface, housed in the proximal end portion 710, the large-diameter portion 730, and the member distal end portion 720. The liquid pipe line 280 is housed in the proximal end portion 710 and the large-diameter portion 730 in a state in which a distal end of the liquid pipe line 280 reaches a distal end edge of the large-diameter portion 730.

Each of the housing part 714 and the housing part 715 has an oval cross-sectional shape perpendicular to the axial direction in portions 714b and 715b, the portions 714b and 715b being provided from the proximal end surface of the proximal end portion 710 to substantially a center of the proximal end portion 710 in the axial direction, and has a perfect circular cross-sectional shape perpendicular to the axial direction in portions 714a and 715a, the portions 714a and 715a being provided from substantially the center of the proximal end portion 710 to the distal end surface of the member distal end portion 720. With such a configuration, on the proximal end side with respect to the distal end portion 7, even in a case where the positions of the fluid pipe line 270 and the through-hole 27k deviate from each other in a state of being viewed in the axial direction, by bending the fluid pipe line 270 in the housing part 715, it is possible to eliminate the deviation of the positions, and it is possible to facilitate a design inside the insertion part 2. Similarly, on the proximal end side with respect to the distal end portion 7, even in a case where the positions of the liquid pipe line 280 and the WJ nozzle 28 deviate from each other in a state of being viewed in the axial direction, by bending the liquid pipe line 280 in the housing part 714, it is possible to eliminate the deviation of the positions, and it is possible to facilitate the design inside the insertion part 2.

As shown in FIG. 11, in the proximal end portion 710, the opening 710a and the outer peripheral surface of the bulging portion 710B are disposed on opposite sides with the center P interposed therebetween. The housing part 711 and the housing part 712 are disposed side by side on a straight line (a straight line LA that passes through the center P and intersects the opening 710a and the bulging portion 710B) connecting the opening 710a and the bulging portion 710B.

The housing part 712 is disposed adjacent to the opening 710a, and is open to the outside of the proximal end portion 710 through the opening 710a. The imaging unit 302 is housed in the housing part 712 not to stay outside the imaginary circle V2.

The housing part 711 is disposed adjacent to the bulging portion 710B, and is disposed between the housing part 712 and the bulging portion 710B. Further, the housing part 711 communicates with the housing part 712.

The housing part 713 is provided adjacent to the housing part 712 and communicates with the housing part 712.

In the proximal end portion 710, the housing part 716 is provided in one of two regions divided by the straight line LA, and the housing part 713 is provided in the other of these two regions. In addition, in the proximal end portion 710, the housing part 715 is provided in one of two regions divided by a straight line LB passing through a center of the housing part 713 and a center of the housing part 716, and the housing part 714 is provided in the other of these two regions.

In FIG. 11, a first reference configuration in which the opening 710a is closed is assumed without changing a disposition of each housing part. In the first reference configuration, it is necessary to secure a thickness of an outer peripheral portion of the proximal end portion 710 that closes the housing part 712. For this reason, it is necessary to move the housing part 712 more toward the center P side to house the imaging unit 302 having a relatively large size. For this movement, it is necessary to reduce a volume of the housing part 711, that is, to reduce an outer diameter of the treatment tool pipe line 260. In addition, in FIG. 11, a second reference configuration in which the bulging portion 710B is not provided is assumed without changing a disposition of each housing part. In the second reference configuration, since it is necessary to form an inner wall of the housing part 711 more inwardly, it is necessary to reduce a size of the housing part 711.

According to a configuration shown in FIG. 11 with respect to these reference configurations, since the housing part 712 can be moved outward, the volume of the housing part 711 can be expanded. Moreover, since there is the bulging portion 710B, the volume of the housing part 711 can be expanded. As a result, the outer diameter of the treatment tool pipe line 260 can be increased without changing the outer diameter of the distal end portion 7. In addition, in the configuration shown in FIG. 11, since the housing part 711 and the housing part 712 communicate with each other, the volume of the housing part 711 can be increased as compared with a case where a wall is formed at a boundary between the housing part 711 and the housing part 712. From this as well, it is possible to increase the outer diameter of the treatment tool pipe line 260. In addition, in the configuration shown in FIG. 11, since the housing part 712 and the housing part 713 communicate with each other, the housing part 712 can be moved more outwardly as compared with a case where a wall is formed at a boundary between the housing part 712 and the housing part 713. From this as well, it is possible to increase the outer diameter of the treatment tool pipe line 260. Further, in the endoscope 1 according to the present aspect, the imaging element 305 is disposed on the radial outer side of the distal end portion 7 in the housing part 712. Therefore, the observation window 30 and the forceps port 26 can be brought close to each other, and a process using the treatment tool can be performed with high accuracy and efficiency.

As described above, according to a configuration of the distal end portion 7 of the endoscope 1 according to the present aspect, it is possible to realize an endoscope in which an inner diameter φ² (see FIG. 7) of the treatment tool pipe line 260 is 3 millimeters (mm) or more, and a ratio of the inner diameter φ² to the outer diameter φ1 (see FIG. 2) of the distal end portion 7 is 35% or more. For example, it is possible to create the endoscope 1 in which a range of the outer diameter φ1 is 7.6 mm or more and 8.5 mm or less and a range of the inner diameter φ2 is 3 mm or more and 3.4 mm or less. In this way, it is possible to realize the endoscope 1 in which the insertion part 2 is thin and the inner diameter of the treatment tool pipe line 260 is large. For a user of the endoscope 1, there is an advantage in that it is easy to insert the endoscope 1 into the subject, and there is an advantage in that various treatment tools can be used in a case where the inner diameter of the treatment tool pipe line 260 is large. For the subject, a burden is small because the insertion part 2 is thin, and a burden is reduced because various operations or examinations can be performed by inserting the endoscope 1 once.

As shown in FIGS. 3 and 8, the fixing member 62 of the bendable portion 6 is fixed to the outer peripheral surface of the proximal end portion 710 while covering the opening 710a of the proximal end portion 710. In this way, the opening 710a is covered with the fixing member 62, and thus, the imaging unit 302 can be protected. Further, as shown in FIG. 3, the fixing member 62 is provided with a notch-shaped opening portion 62A at a position corresponding to the bulging portion 710B. By exposing the bulging portion 710B from the opening portion 62A, it is possible to prevent the distal end portion 7 from becoming thick. As shown in FIGS. 3 and 8, a distal end of the fixing member 62 abuts against a proximal end surface of the large-diameter portion 730 (a region between the imaginary circle V2 and the outer peripheral edge of the large-diameter portion 730 in FIG. 11), and accordingly, the bendable portion 6 and the distal end portion 7 are positioned in the axial direction.

In FIG. 12, an imaginary circle V3 similar to the imaginary circle described in FIG. 11 is shown. The imaginary circle V3 is concentric with the imaginary circle V2, but has a different diameter. The member distal end portion 720 comprises a body part 720A located inside the imaginary circle V3, and a bulging portion 720B that bulges from the body part 720A to the outside of the imaginary circle V3. An outer peripheral edge of the body part 720A has a shape overlapping with an outer peripheral edge of the imaginary circle V3, and an outer peripheral edge of the bulging portion 720B has a shape that bulges to the outside of the imaginary circle V3. On an outer peripheral surface of the body part 720A, the distance from the center P is a distance L3 that is the same as a radius of the imaginary circle V3, and on an outer peripheral surface of the bulging portion 720B, the distance from the center P is a distance L4 that is larger than the distance L3. The bulging portion 720B is adjacent to the housing part 715, and the bulging portion 720B is provided along an outer peripheral edge of the housing part 715. By providing the bulging portion 720B, the housing part 715 can be disposed at a position farther from the housing part 712 without increasing the diameter of the distal end portion 7. Therefore, it is possible to sufficiently secure an installation space for the nozzle 27 mounted on the housing part 715.

In addition, as shown in FIGS. 10 and 12, on the outer peripheral surface of the body part 720A on a distal end side, a notch 716A is formed at a position corresponding to the housing part 716, and a notch 713A is formed at a position corresponding to the housing part 713. By providing the notch 713A and the notch 716A, the housing part 713 and the housing part 716 can be disposed more outward, so that a size of the housing part 711 can be sufficiently secured.

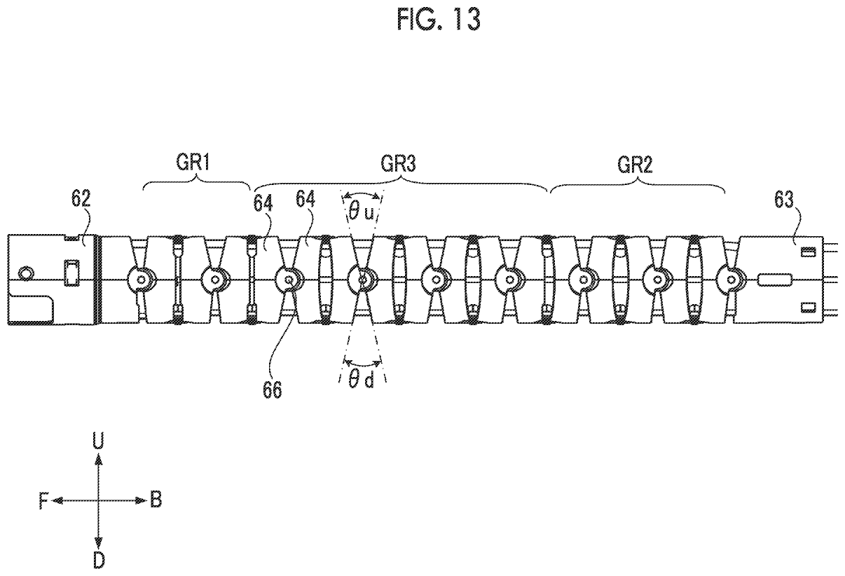

FIG. 13 is a side view of the bending piece portion 60 as viewed in the right direction. In FIG. 13, a distal end direction F that is a direction from the proximal end to the distal end of the insertion part 2, and a proximal end direction B that is opposite to the distal end direction F are shown.

The plurality of annular members 64 included in the bending piece portion 60 are grouped into a distal end group GR1 located on a distal end side in the axial direction, a proximal end group GR2 located on a proximal end side in the axial direction, and an intermediate group GR3 located between the distal end group GR1 and the proximal end group GR2. In an example of FIG. 13, the first to third annular members 64 counted from the distal end side constitute the distal end group GR1. In addition, the first to fifth annular members 64 counted from the proximal end side constitute the proximal end group GR2. In addition, the annular members 64 counted from the fourth annular member 64 counted from the distal end side to the sixth annular member 64 counted from the proximal end side constitute the intermediate group GR3.

Each annular member 64 belonging to these groups is rotatable around an axis extending in the left-right direction. A maximum rotation angle θd at which a lower portion of the annular members 64 can be rotated with respect to a straight line connecting all the caulking pins 66 as a boundary is the largest in the intermediate group GR3, and is the smallest in the distal end group GR1, and in the proximal end group GR2, the maximum rotation angle θd is larger than that in the distal end group GR1 and is smaller than that in the intermediate group GR3. With a configuration in which an average value of the maximum rotation angle θd is 180 or more, it is possible to increase a bending angle in the lower direction. Similarly, a maximum rotation angle θu at which an upper portion of the annular members 64 can be rotated with respect to the straight line connecting all the caulking pins 66 as a boundary is the largest in the intermediate group GR3, and is the smallest in the distal end group GR1, and in the proximal end group GR2, the maximum rotation angle θu is larger than that in the distal end group GR1 and is smaller than that in the intermediate group GR3. Even at the maximum rotation angle θu, it is possible to increase a bending angle in the upper direction by setting an average value of the maximum rotation angle θu to 22° or more. For example, it is possible to realize the insertion part 2 in which a bending angle of the bendable portion 6 in the upper direction is 210° and a bending angle of the bendable portion 6 in the lower direction is 160°. Further, according to this configuration, since the distal end group GR1 is not largely bent, a fixing state between the distal end portion 7 and the incorporated object group OB can be stably maintained. In addition, the intermediate group GR3 is bent to be the largest, thereby contributing to an improvement of the bending angle. Further, since the proximal end group GR2 is less likely to be bent than the intermediate group GR3, it is possible to bend the bendable portion 6 without a local concentration of stress during bending of the bendable portion 6.

The bending piece portion 60 includes sixteen annular members 64 in total, but all of these do not need to have the same shape. For example, as the sixteen annular members 64, it is preferable to use a plurality of types of the annular members 64 in which inclination angles of a proximal end-side end surface and a distal end-side end surface with respect to the up-down direction are different from each other. The bending piece portion 60 is preferably configured by using eight or more types of the annular members 64, and a total number thereof is preferably 16. With this configuration, it is possible to realize the insertion part 2 in which the bending angle of the bendable portion 6 in the upper direction is 210° and the bending angle of the bendable portion 6 in the lower direction is 160°.

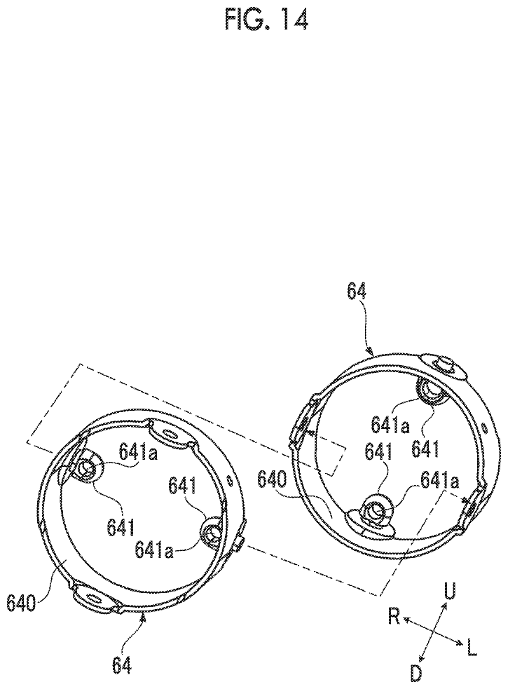

FIG. 14 is an exploded perspective view of two adjacent annular members 64 in the bending piece portion 60. A pair of insertion members 641 including insertion holes 641a into which the wire W is inserted are provided in one annular member 64 to face each other in the left-right direction. In the other annular member 64, a pair of insertion members 641 including insertion holes 641a into which the wire W is inserted are provided to face each other in the up-down direction. The insertion member 641 is provided to protrude from an inner peripheral surface 640 of the annular member 64 in a state of being viewed in the axial direction. The insertion member 641 functions as a guide member for the wire W.

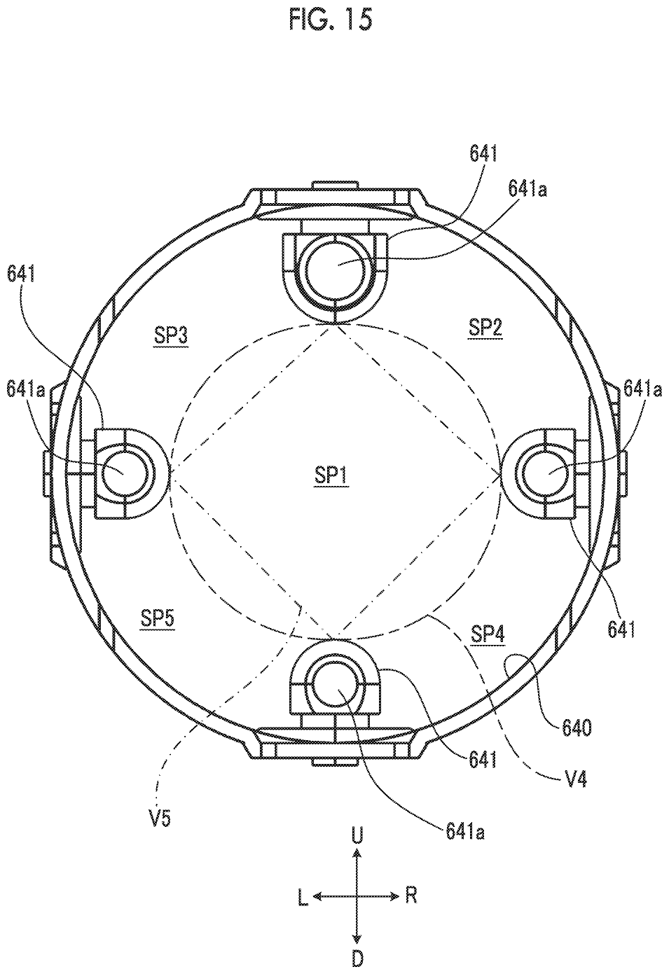

FIG. 15 is a view of a connection state of the two annular members 64 shown in FIG. 14 as viewed from the proximal end side in the axial direction. As shown in FIG. 15, in a state of being viewed in the axial direction, inside the bending piece portion 60, four insertion members 641 are disposed at different positions in a circumferential direction of the annular member 64.

An inner space of the bending piece portion 60 in a state of being viewed in the axial direction is divided into a space SP1, a space SP2, a space SP3, a space SP4, and a space SP5. The space SP1 constitutes the first space, and the space SP2 to the space SP5 constitute the second space.

The space SP1 is a space partitioned by an inscribed circle V4 inscribed in the four insertion members 641. In FIG. 15, a quadrangle V5 formed by connecting vertices of the insertion members 641 adjacent to each other in the circumferential direction is shown. The inscribed circle V4 can also be referred to as a circumscribed circle of the quadrangle V5. The inscribed circle V4 may be a perfect circle or an ellipse.

The space SP2 is a space partitioned by the insertion member 641 on a right side in FIG. 15, the insertion member 641 on an upper side in FIG. 15, the space SP1, and the inner peripheral surface 640.

The space SP3 is a space partitioned by the insertion member 641 on a left side in FIG. 15, the insertion member 641 on the upper side in FIG. 15, the space SP1, and the inner peripheral surface 640.

The space SP4 is a space partitioned by the insertion member 641 on the right side in FIG. 15, the insertion member 641 on a lower side in FIG. 15, the space SP1, and the inner peripheral surface 640.

The space SP5 is a space partitioned by the insertion member 641 on the left side in FIG. 15, the insertion member 641 on the lower side in FIG. 15, the space SP1, and the inner peripheral surface 640.

A circumferential distance between two insertion members 641 adjacent to each other in the circumferential direction of the annular member 64 among the four insertion members 641 in an orthographic projection (a state shown in FIG. 15) viewed in the axial direction is preferably 1.3 times a distance between the vertices of the two insertion members 641 in the inscribed circle V4.

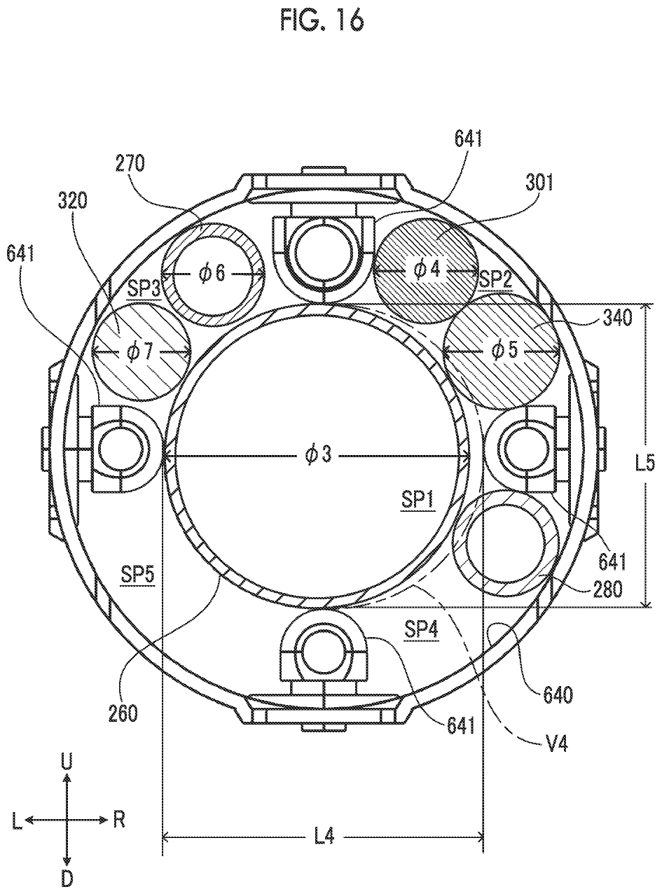

FIG. 16 is a schematic view showing a disposition of incorporated objects that are incorporated in the bending piece portion 60. As shown in FIG. 16, the incorporated object group OB (the treatment tool pipe line 260, the fluid pipe line 270, the liquid pipe line 280, the cable 301, the left light guide 320, and the right light guide 340) described above is housed in the inner space of the bending piece portion 60. In FIG. 16, each incorporated object included in the incorporated object group OB is shown in a cross section perpendicular to the axial direction. In addition, in FIG. 16, the wire W included in the incorporated object group OB is not shown. Each of the treatment tool pipe line 260, the fluid pipe line 270, the liquid pipe line 280, the cable 301, the left light guide 320, and the right light guide 340 has a circular outer shape of the cross section perpendicular to the axial direction in the bendable portion 6.

As shown in FIG. 16, each of the treatment tool pipe line 260, the fluid pipe line 270, and the liquid pipe line 280 is a hollow tubular incorporated object in at least the bendable portion 6. The hollow incorporated object refers to an incorporated object in which a ratio of a space to an area of an outer peripheral circle is 20% or more in the cross section perpendicular to the axial direction. Since a large number of signal lines are present inside the cable 301, the cable 301 can be referred to as a non-hollow incorporated object. Since a large number of optical fibers are present in each of the left light guide 320 and the right light guide 340, the left light guide 320 and the right light guide 340 can be referred to as non-hollow incorporated objects.

As shown in FIG. 16, the treatment tool pipe line 260, which is a hollow incorporated object, is disposed in the space SP1. The treatment tool pipe line 260 has the largest outer diameter in the incorporated object group OB. The treatment tool pipe line 260 has a size that fills almost the entire space SP1. That is, a difference between the distances L4 and L5 between the two insertion members 641 facing each other and an outer diameter φ3 of the treatment tool pipe line 260 is sufficiently small, and is, for example, equal to or less than half of an outer diameter of the wire W, which is the incorporated object having the smallest outer diameter included in the bendable portion 6. In addition, in a state of being viewed in the axial direction, a distance between a center of the annular member 64 and a center of the treatment tool pipe line 260 is sufficiently small, and is, for example, equal to or less than half of the outer diameter of the wire W.

The cable 301, which is a non-hollow incorporated object, and the right light guide 340, which is a non-hollow incorporated object, are disposed in the space SP2. The cable 301 has higher rigidity than the right light guide 340. The cable 301 is disposed above the right light guide 340. The cable 301 is maintained in a state of being in contact with the upper insertion member 641, the right light guide 340, and the inner peripheral surface 640. The right light guide 340 is maintained in a state of being in contact with the right insertion member 641, the cable 301, and the inner peripheral surface 640.

A difference between an outer diameter φ4 of the cable 301 and an outer diameter φ5 of the right light guide 340 is sufficiently small, and is, for example, equal to or less than half of the outer diameter of the wire W. With this configuration, it is possible to eliminate an empty space in the space SP2 as much as possible while minimizing an amount by which at least one of the cable 301 or the right light guide 340 protrudes into the space SP1. Therefore, it is possible to prevent the treatment tool pipe line 260 disposed in the space SP1 from entering the space SP2 in a case where the bendable portion 6 is bent, or the like. In addition, it is possible to maximize the outer diameter of the treatment tool pipe line 260. Further, both the cable 301 and the right light guide 340 have high rigidity. The high rigidity can also prevent the treatment tool pipe line 260 disposed in the space SP1 from entering the space SP2.

The fluid pipe line 270, which is a hollow incorporated object, and the left light guide 320, which is a non-hollow incorporated object, are disposed in the space SP3. The fluid pipe line 270 is disposed closer to the cable 301 than the left light guide 320. Accordingly, it is easy to bring the nozzle 27 and the observation window 30 close to each other on the distal end surface 14.

The left light guide 320 has lower rigidity than the right light guide 340. A difference in rigidity is obtained due to a difference in a type, the number of layers, a layer thickness, and the like of the coating member. That is, an outer diameter φ7 of the left light guide 320 is smaller than the outer diameter φ5 of the right light guide 340. For example, the right light guide 340 and the left light guide 320 have different numbers of coating members for coating the outer surfaces thereof. Specifically, the right light guide 340 is obtained by further providing the coating member on an outer periphery of the left light guide 320.

A difference between an outer diameter φ6 of the fluid pipe line 270 and the outer diameter φ7 of the left light guide 320 is sufficiently small, and is, for example, equal to or less than half of the outer diameter of the wire W.

With this configuration, in a case where the space SP2 and the space SP3 are compared with each other, the empty space of the space SP3 is larger. That is, a sum of the outer diameter φ6 of the fluid pipe line 270 and the outer diameter φ7 of the left light guide 320 is smaller than a sum of the outer diameter φ4 of the cable 301 and the outer diameter φ5 of the right light guide 340. Accordingly, even in a case where the treatment tool pipe line 260 disposed in the space SP1 is brought close to the space SP3, a space in which the fluid pipe line 270 and the left light guide 320 escape can be secured. By securing such a space, it is possible to maximize the outer diameter of the treatment tool pipe line 260.

The fluid pipe line 270 has the same outer diameter as that of the cable 301, the right light guide 340, and the left light guide 320, but is hollow, and thus has lower rigidity than the cable 301, the right light guide 340, and the left light guide 320. Accordingly, regarding the incorporated object disposed in the space SP3 and the incorporated object disposed in the space SP2, the incorporated object disposed in the space SP3 is relatively more likely to be deformed. For this reason, even in a case where the treatment tool pipe line 260 is brought close to the space SP3 and is in contact with the fluid pipe line 270 and the left light guide 320, an empty space is formed by deformation or movement of the fluid pipe line 270 and the left light guide 320 themselves, whereby the treatment tool pipe line 260 can escape.

The hollow liquid pipe line 280 is disposed in the space SP4. Since the liquid pipe line 280 is disposed in the space SP4, it is possible to restrain the treatment tool pipe line 260 from moving in the space SP1.

As described above, in the endoscope 1, in the bendable portion 6, the cable 301, the right light guide 340, the fluid pipe line 270, the left light guide 320, and the liquid pipe line 280 are disposed around the treatment tool pipe line 260. In other words, in a state of being viewed in the axial direction, a distance between the center of the annular member 64 and a center of the incorporated objects (each of the cable 301, the right light guide 340, the fluid pipe line 270, the left light guide 320, and the liquid pipe line 280) other than the treatment tool pipe line 260 is larger than the distance between the center of the annular member 64 and the center of the treatment tool pipe line 260. In the space SP2, which is one of four spaces SP2 to SP5 surrounding the treatment tool pipe line 260, the cable 301 and the right light guide 340 having high rigidity and the same outer diameter are disposed such that the treatment tool pipe line 260 does not move, and in the space SP3 adjacent to the space SP2, the fluid pipe line 270 and the left light guide 320 having low rigidity and the same outer diameter are disposed so that a large amount of empty space is secured. Accordingly, it is possible to realize an endoscope in which the inner diameter φ2 of the treatment tool pipe line 260 is 3 mm or more and the ratio of the inner diameter φ2 to the outer diameter φ1 of the distal end portion 7 is 35% or more.

As described above, the following matters are disclosed in the present specification.

Although the constituent elements and the like corresponding to the above embodiments are shown in parentheses, the present invention is not limited thereto.

(1)

An endoscope comprising:
an insertion part (insertion part 2),
in which the insertion part incorporates a treatment tool pipe line (treatment tool pipe line 260) into which a treatment tool is inserted, an imaging unit (imaging unit 302), and a part of a cable (cable 301) connected to the imaging unit,
a distal end portion (distal end portion 7) of the insertion part has a tubular first member (first member 71) that houses an incorporated object of the insertion part,
a first housing part (housing part 711) that houses the treatment tool pipe line and a second housing part (housing part 712) that houses the imaging unit are provided adjacent to each other at a proximal end portion (proximal end portion 710) of the first member,
an opening (opening 710a) communicating with the second housing part is formed on an outer peripheral surface of the proximal end portion,
in a state of being viewed in an axial direction of the insertion part, the outer peripheral surface of the proximal end portion includes a first region (outer peripheral surface of body part 710A) where a distance from a center (center P) of the insertion part is a first distance (distance L1), and a second region (outer peripheral surface of bulging portion 710B) where the distance is a second distance (distance L2) that is larger than the first distance,
in a state of being viewed in the axial direction, the second region and the opening are disposed on opposite sides with the center of the insertion part interposed therebetween, and
the first housing part is disposed adjacent to the second region.

(2)

An endoscope comprising:
an insertion part (insertion part 2),
in which the insertion part incorporates a treatment tool pipe line (treatment tool pipe line 260) into which a treatment tool is inserted, and
a ratio of an inner diameter (inner diameter φ2) of the treatment tool pipe line to an outer diameter (outer diameter φ1) of a distal end portion (distal end portion 7) of the insertion part is 35% or more, and the inner diameter is 3 mm or more.

(3)

The endoscope according to (1),
in which the first housing part and the second housing part communicate with each other.

(4)

The endoscope according to (3),
in which a third housing part (housing part 713) that houses the incorporated object is provided at the proximal end portion, and
the second housing part and the third housing part communicate with each other.

(5)

The endoscope according to (4),
in which the incorporated object housed in the third housing part is a light guide (right light guide 340).

(6)

The endoscope according to any one of (1), and (3) to (5),
in which a fourth housing part (housing part 714, 715) that houses the incorporated object is provided at the proximal end portion, and
a proximal end side of the fourth housing part has an oval cross section perpendicular to the axial direction.

(7)

The endoscope according to (6),
in which the incorporated object housed in the fourth housing part is a fluid pipe line (fluid pipe line 270, liquid pipe line 280).

(8)

The endoscope according to any one of (1), and (3) to (5),
in which a fourth housing part (housing part 715) that houses a fluid pipe line (fluid pipe line 270) in which a nozzle (nozzle 27) is fixed to a distal end thereof, as the incorporated object, is provided at the proximal end portion,
the distal end portion of the insertion part has a second member (second member 72) fixed to an outer peripheral surface of the first member on a distal end side,
in a state of being viewed in the axial direction, the outer peripheral surface of the first member that is fixed to the second member includes a third region (outer peripheral surface of body part 720A) where a distance from the center of the insertion part is a third distance (distance L3), and a fourth region (outer peripheral surface of bulging portion 720B) where the distance is a fourth distance (distance L4) that is larger than the third distance, and
the fourth region is provided along an outer peripheral edge of the fourth housing part.

(9)

The endoscope according to any one of (1), and (3) to (8),
in which the insertion part includes a bendable portion (bendable portion 6) that has a fixing member (fixing member 62) fixed to the outer peripheral surface of the proximal end portion, at a distal end thereof, and
the fixing member covers the opening and has an opening portion (opening portion 62A) that exposes the second region.

(10)

The endoscope according to any one of (1), and (3) to (9),
in which the first member has a convex portion (large-diameter portion 730) extending on an outer peripheral surface between a distal end and a proximal end along a circumferential direction, and
the second region is provided from a proximal end side of the convex portion to a proximal end surface of the first member.

(11)

The endoscope according to (10),
in which the insertion part includes a bendable portion (bendable portion 6) that has a fixing member (fixing member 62) fixed to the outer peripheral surface of the proximal end portion, at a distal end thereof, and
a distal end of the fixing member abuts against the convex portion.

(12)

The endoscope according to any one of (1), and (3) to (11),
in which the proximal end portion includes a housing part (housing part 716) in which a first light guide (left light guide 320) is housed, a housing part (housing part 713) in which a second light guide (right light guide 340) is housed, a housing part (housing part 715) in which a first fluid pipe line (fluid pipe line 270) is housed, and a housing part (housing part 714) in which a second fluid pipe line (liquid pipe line 280) is housed,
in a state of being viewed in the axial direction, the housing part in which the first light guide is housed is provided in one of two regions of the proximal end portion, and the housing part in which the second light guide is housed is provided in the other of the two regions, the two regions being divided by a straight line (straight line LA) connecting the opening, the center of the insertion part, and the second region, and
in a state of being viewed in the axial direction, the housing part in which the first fluid pipe line is housed is provided in one of two regions of the proximal end portion, and the housing part in which the second fluid pipe line is housed is provided in the other of the two regions, the two regions being divided by a straight line (straight line LB) passing through a center of the housing part in which the first light guide is housed and a center of the housing part in which the second light guide is housed.

(13)

The endoscope according to any one of (1), and (3) to (12),
in which the second housing part houses the imaging unit, and
a rear surface of an imaging element included in the imaging unit is disposed to face a radial outer side of the distal end portion.

### EXPLANATION OF REFERENCES

1: endoscope
2: insertion part
3: operating part
4: universal cord
5: soft portion
6: bendable portion
7: distal end portion
8, 9: angle knob
10: air/water supply button
11: suction button
12: treatment tool inlet port
14, 302A, 320A, 340A: distal end surface
14A, 14B: inclined surface
20: first surface
21: second surface
21A, 22A: flat surface
21B, 22B: stepped portion
22: third surface
26: forceps port
27: nozzle
27A: jetting port
27k, 300k, 320k, 340k: through-hole
28: WJ nozzle
30: observation window
32: first illumination window
34: second illumination window
60: bending piece portion
61: coating member
62: fixing member
62A: opening portion
63: connecting member
64: annular member
66: caulking pin
71: first member
710a: opening
72: second member
260: treatment tool pipe line
270: fluid pipe line
280: liquid pipe line
301: cable
302: imaging unit (imager)
303: lens group
304: prism
305: imaging element
306: main board
307: sub-board
308: strand
320: left light guide
340: right light guide
640: inner peripheral surface
641: insertion member
641a: insertion hole
710: proximal end portion
710A, 720A: body part
710B, 720B: bulging portion
711, 712, 713, 714, 715, 716: housing part
714a, 714b, 715a, 715b: portion
720: member distal end portion
730: large-diameter portion
V1, V2, V3: imaginary circle
GR1: distal end group
GR2: proximal end group
GR3: intermediate group
V4: inscribed circle
V5: quadrangle
SP1, SP2, SP3, SP4, SP5: space
L1, L2, L3, L4: distance

What is claimed is:

1. An endoscope comprising:
an insertion part,
wherein the insertion part incorporates a treatment tool pipe line into which a treatment tool is to be inserted, an imager, and a part of a cable connected to the imager,
a distal end portion of the insertion part has a tubular first member that houses an incorporated object of the insertion part,
a first housing part that houses the treatment tool pipe line and a second housing part that houses the imager are provided adjacent to each other at a proximal end portion of the first member,
an opening communicating with the second housing part is formed at an outer peripheral surface of the proximal end portion,
in a state of being viewed in an axial direction of the insertion part, the outer peripheral surface of the proximal end portion includes a first region where a distance from a center of the insertion part is a first distance, and a second region where the distance is a second distance that is larger than the first distance,
in a state of being viewed in the axial direction, the second region and the opening are disposed on opposite sides with the center of the insertion part interposed therebetween, and
the first housing part is disposed adjacent to the second region.

2. An endoscope comprising:
an insertion part,
wherein the insertion part incorporates a treatment tool pipe line into which a treatment tool is to be inserted, and
a ratio of an inner diameter of the treatment tool pipe line to an outer diameter of a distal end portion of the insertion part is 35% or more, and the inner diameter is 3 mm or more.

3. The endoscope according to claim 1,
wherein the first housing part and the second housing part communicate with each other.

4. The endoscope according to claim 3,
wherein a third housing part that houses the incorporated object is provided at the proximal end portion, and
the second housing part and the third housing part communicate with each other.

5. The endoscope according to claim 4,
wherein the incorporated object housed in the third housing part is a light guide.

6. The endoscope according to claim 1,
wherein a fourth housing part that houses the incorporated object is provided at the proximal end portion, and
a proximal end side of the fourth housing part has an oval cross section perpendicular to the axial direction.

7. The endoscope according to claim 6,
wherein the incorporated object housed in the fourth housing part is a fluid pipe line.

8. The endoscope according to claim 1,
wherein a fourth housing part that houses a fluid pipe line to a distal end of which a nozzle is fixed, as the incorporated object, is provided at the proximal end portion,
the distal end portion of the insertion part has a second member fixed to an outer peripheral surface of the first member on a distal end side of the first member,
in a state of being viewed in the axial direction, the outer peripheral surface of the first member that is fixed to the second member includes a third region where a distance from the center of the insertion part is a third distance, and a fourth region where the distance is a fourth distance that is larger than the third distance, and
the fourth region is provided along an outer peripheral edge of the fourth housing part.

9. The endoscope according to claim 1,
wherein the insertion part includes a bendable portion that has a fixing member fixed to the outer peripheral surface of the proximal end portion, at a distal end of the bendable portion, and
the fixing member covers the opening and has an opening portion that exposes the second region.

10. The endoscope according to claim 1,
wherein the first member has a convex portion extending on an outer peripheral surface of the first member between a distal end and a proximal end the first member along a circumferential direction of the first member, and
the second region is provided from a proximal end side of the convex portion to a proximal end surface of the first member.

11. The endoscope according to claim 10,
wherein the insertion part includes a bendable portion that has a fixing member fixed to the outer peripheral surface of the proximal end portion, at a distal end of bendable portion, and
a distal end of the fixing member abuts against the convex portion.

12. The endoscope according to claim 1,
wherein the proximal end portion includes a housing part in which a first light guide is housed, a housing part in which a second light guide is housed, a housing part in which a first fluid pipe line is housed, and a housing part in which a second fluid pipe line is housed,
in a state of being viewed in the axial direction, the housing part in which the first light guide is housed is provided in one of two regions of the proximal end portion, and the housing part in which the second light guide is housed is provided in other of the two regions, the two regions being divided by a virtual straight line connecting the opening, the center of the insertion part, and the second region, and
in a state of being viewed in the axial direction, the housing part in which the first fluid pipe line is housed is provided in one of two regions of the proximal end portion, and the housing part in which the second fluid pipe line is housed is provided in other of the two regions, the two regions being divided by a virtual straight line passing through a center of the housing part in which the first light guide is housed and a center of the housing part in which the second light guide is housed.

13. The endoscope according to claim 1,
wherein the second housing part houses the imager, and
a rear surface of an imaging element included in the imager is disposed to face a radial outer side of the distal end portion.

14. The endoscope according to claim 2,
wherein the outer diameter of the distal end portion is 7.6 mm or more and 8.5 mm or less.
